(19) United States
(12) Patent Application Publication
(10) Pub. No.: US 2012/0197123 A1
(43) Pub. Date: Aug. 2, 2012

(54) Systems and Methods for Determining Global Circumferential Strain in Cardiology

(75) Inventors: Morris Ziv-Ari, Tirat Hacarmel (IL); Zvi Friedman, Tirat Hacarmel (IL); Peter Lysyansky, Tirat Hacarmel (IL); Nahum Smirin, Haifa (IL)

(73) Assignee: General Electric Company, Schenectady, NY (US)

(21) Appl. No.: 13/017,635

(22) Filed: Jan. 31, 2011

(51) Int. Cl. A61B 8/08

(52) U.S. Cl. 600/443

(57) ABSTRACT

Systems and methods for determining circumference and a percent change in circumference of a structure are provided. Certain methods include receiving first and second sets of at least three vertical plane images of a structure about a long axis of the structure that were acquired at first and second points in time, respectively. Data points from each of the images of each of the sets that define a contour of the structure are identified. The circumference of the structure at a horizontal plane of the structure at the first and second points in time using identified data points positioned on the horizontal plane is estimated. A percent change between the estimated circumference of the structure at the first point in time and the estimated circumference of the structure at the second point in time is determined.

### RELATED APPLICATIONS

[Not Applicable]

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

Embodiments of the present technology generally relate to cardiology. More specifically, embodiments of the present technology provide for determining global circumferential strain (“GCS”) in the left ventricle of a heart.

GCS is a parameter for echocardiographic quantification of myocardial (dys)function. When the heart pumps, and the left ventricle contracts, the circumference of the left ventricle varies. GCS is the relative change between a first circumference of the left ventricle and a second circumference of the left ventricle.

Known techniques of determining GCS include comparing two dimensional (“2D”) scans taken along the short axis (horizontal plane) of a left ventricle. 2D scanning can provide high temporal-spatial sampling rates resulting in robust two dimensional speckle tracking and the possibility to track fast cardiac mechanical events. However, comparing 2D scans using known techniques can be time consuming and can require a high level of skill by the technician that is performing the scan. Also, 2D scans taken along the short axis of a left ventricle are generally taken at three scan planes: the apex (AP) plane located at the apex of the left ventricle, the papillary muscles (PM) plane located at the papillary muscles of the left ventricle, and the mitral valve (MV) plane located at the mitral valve of the left ventricle.

Known techniques of determining GCS also include comparing four dimensional (“4D”) scans taken along the short axis (horizontal plane) of a left ventricle. However, such techniques can provide inaccurate tracking due to low frame rate at which the scan is taken. Also, such techniques can have limited spatial and temporal resolution. 4D scanning can provide short axis planes at any level of the left ventricle, rather than being limited to three levels like 2D scanning. However, analysis of 4D scans can be inaccurate.

Thus, there is a need for improved systems and methods for determining GCS in the left ventricle of a heart.

### BRIEF SUMMARY OF THE INVENTION

Embodiments of the present technology provide a method for determining circumference and a percent change in circumference of a structure comprising: receiving at a processor a first set of at least three vertical plane images of a structure about a long axis of the structure that were acquired at a first point in time; using the processor to identify data points from each of the images of the first set that define a contour of the structure; using the processor to estimate circumference of the structure at a horizontal plane of the structure at the first point in time using identified data points positioned on the horizontal plane; receiving at the processor a second set of at least three vertical plane images of a structure about a long axis of the structure that were acquired at a second point in time; using a processor to identify data points from each of the images of the second set that define a contour of the structure; using the processor to estimate circumference of the structure at the horizontal plane of the structure at the second point in time using identified data points positioned on the horizontal plane; and using the processor determine the percent change between the estimated circumference of the structure at the first point in time and the estimated circumference of the structure at the second point in time.

In an embodiment, the structure is a left ventricle and the percent change between the circumference of the structure at the first point in time and at the second point in time is a global circumferential strain for the left ventricle.

Certain embodiments further include displaying the global circumferential strain as a color map.

Certain embodiments further include displaying the global circumferential strain as a line chart.

Certain embodiments further include adjusting at least one of the estimated circumferences of the structure using a linear drift correction that assumes the circumference of the left ventricle is the same at the beginning and end of each heart pump cycle.

In an embodiment, the first point in time is the beginning of systole, and wherein the second point in time is the end of systole.

In an embodiment, the vertical plane images are acquired using an ultrasound scanner.

Embodiments of the present technology provide a system for determining circumference and a percent change in circumference of a structure comprising: a processor configured to receive a first set of at least three vertical plane images of a structure about a long axis of the structure that were acquired at a first point in time, the processor configured to identify data points from each of the images of the first set that define a contour of the structure, the processor configured to estimate circumference of the structure at a horizontal plane of the structure at the first point in time using identified data points positioned on the horizontal plane, the processor configured to receive a second set of at least three vertical plane images of a structure about a long axis of the structure that were acquired at a second point in time, the processor configured to identify data points from each of the images of the second set that define a contour of the structure, the processor configured to estimate circumference of the structure at the horizontal plane of the structure at the second point in time using identified data points positioned on the horizontal plane, and the processor configured to determine the percent change between the estimated circumference of the structure at the first point in time and the estimated circumference of the structure at the second point in time.

In an embodiment, the structure is a left ventricle and the percent change between the circumference of the structure at the first point in time and at the second point in time is a global circumferential strain for the left ventricle.

Certain embodiments further include an output device configured to display the global circumferential strain as a color map.

Certain embodiments further include an output device configured to display the global circumferential strain as a line chart.

In an embodiment, the processor is configured to adjust at least one of the estimated circumferences of the structure using a linear drift correction that assumes the circumference of the left ventricle is the same at the beginning and end of each heart pump cycle.

In an embodiment, the first point in time is the beginning of systole, and wherein the second point in time is the end of systole.

Certain embodiments further include an ultrasound scanner configured to acquire the vertical plane images.

Embodiments of the present technology provide a non-transitory computer-readable medium encoded with a set of instructions for a computer, the instructions comprising: a first routine configured to receive a first set of at least three vertical plane images of a structure about a long axis of the structure that were acquired at a first point in time; a second routine configured to identify data points from each of the images of the first set that define a contour of the structure; a third routine configured to estimate circumference of the structure at a horizontal plane of the structure at the first point in time using identified data points positioned on the horizontal plane; a fourth routine configured to receive a second set of at least three vertical plane images of a structure about a long axis of the structure that were acquired at a second point in time; a fifth routine configured to identify data points from each of the images of the second set that define a contour of the structure; a sixth routine configured to estimate circumference of the structure at the horizontal plane of the structure at the second point in time using identified data points positioned on the horizontal plane; and a seventh routine configured to determine the percent change between the estimated circumference of the structure at the first point in time and the estimated circumference of the structure at the second point in time.

In an embodiment, the structure is a left ventricle and the percent change between the circumference of the structure at the first point in time and at the second point in time is a global circumferential strain for the left ventricle.

Certain embodiments further include an eighth routine configured to display the global circumferential strain as a color map.

Certain embodiments further include an eighth routine configured to display the global circumferential strain as a line chart.

Certain embodiments further include an eighth routine configured to adjust at least one of the estimated circumferences of the structure using a linear drift correction that assumes the circumference of the left ventricle is the same at the beginning and end of each heart pump cycle.

In an embodiment, the first point in time is the beginning of systole, and wherein the second point in time is the end of systole.

### BRIEF DESCRIPTION OF THE DRAWINGS

FIG. 1 depicts scans taken along the short axis (horizontal plane) of a left ventricle at the level of the papillary muscles, and related analysis, as is known in the art.

FIG. 2 depicts three 2D ultrasound scans taken along the short axis (horizontal plane) of a left ventricle.

FIG. 3 depicts three 2D ultrasound scans taken along the long axis (vertical plane) of a left ventricle.

FIG. 4 depicts data derived from three 2D scans taken along the long axis (vertical plane) of a left ventricle in accordance with embodiments of the present technology.

FIG. 5 is a top view of the data depicted in FIG. 4.

FIG. 6 depicts a model of horizontal plane circumferences of a left ventricle based on the data depicted in FIG. 4.

FIG. 7 depicts a geometric model for use in calculating the circumference of an ellipse used in accordance with embodiments of the present technology.

FIG. 8 depicts a technique for correcting circumference estimates in accordance with embodiments of the present technology.

FIG. 9 depicts a color map that provides the circumference of a left ventricle over time in accordance with embodiments of the present technology.

FIG. 10 depicts a color map that provides the GCS of a left ventricle over time in accordance with embodiments of the present technology.

FIG. 11 depicts a comparison of GCS values determined using a known technique to GCS values determined using a technique in accordance with an embodiment of the present technology.

FIG. 12 depicts a system for determining GCS in accordance with embodiments of the present technology.

FIG. 13 depicts a method for determining GCS in accordance with embodiments of the present technology.

The foregoing summary, as well as the following detailed description of certain embodiments, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF CERTAIN EMBODIMENTS

Embodiments of the present technology generally relate to cardiology. More specifically, embodiments of the present technology provide for determining global circumferential strain (“GCS”) in the left ventricle of a heart.

While embodiments described herein are in the context of cardiology, and using ultrasound imaging techniques to determine GCS, in certain embodiments, the techniques described herein can be used to determine the circumferential variance of any structure that is imaged using any imaging technology.

It has been found that two-dimensional (“2D”) scans taken along the long axis (vertical plane) of a structure, such as the left ventricle, for example, can be used to determine GCS at any horizontal plane of the structure. It has been found that determining GCS using such techniques, as described herein, can overcome certain limitations associated with determining GCS using known techniques.

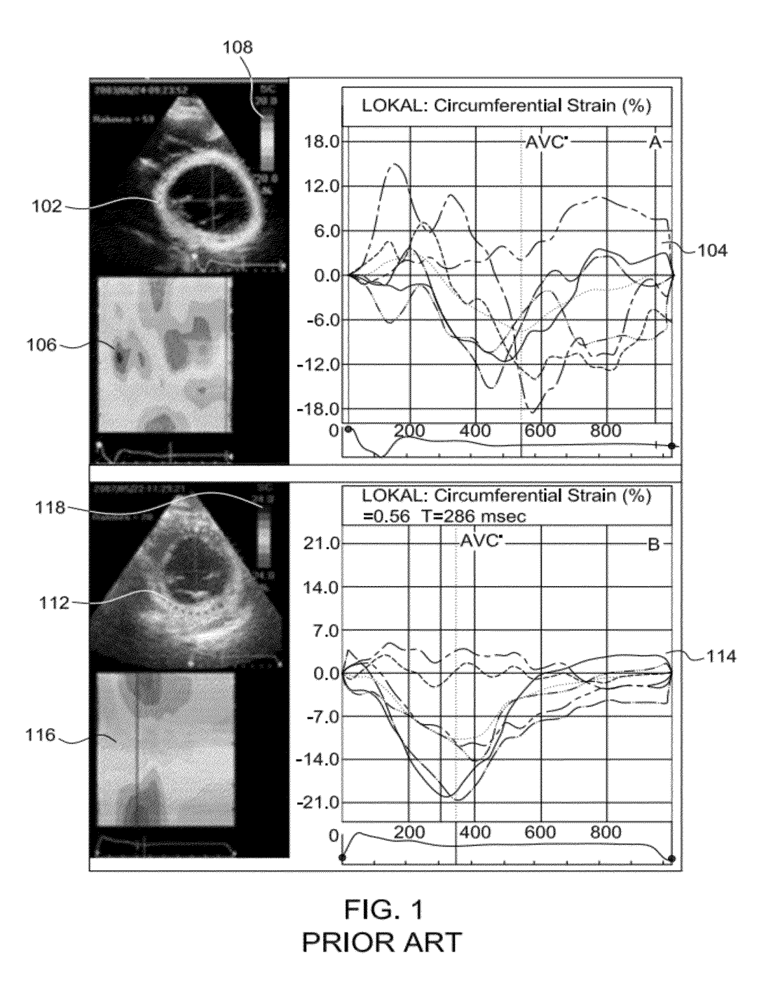

FIG. 1 depicts scans taken along the short axis (horizontal plane) of a left ventricle at the level of the papillary muscles, and related GCS analysis, as is known in the art.

As depicted in FIG. 1, the first ultrasound scan 102 shows the left ventricle with asynchronous circumferential contraction with postsystolic shortening and passive movement of the inferior and posterior segments, which are scar tissue. GCS for the ventricle is depicted as a function of time on graph 104 by dashed white line. The solid lines on graph 104 are the local circumferential strains around the short axis cross section. Local Circumferential Strains for the left ventricle at the level in which the short axis plane was acquired are depicted as a spatial color map 106 with the colors being defined in color key 108.

As also depicted in FIG. 1, the second ultrasound scan 112 shows the same left ventricle after four years of Cardiac Resynchronization Therapy (“CRT”), which has been known to improve GCS. The second scan 112 shows increased synchronous contraction, a reduction of postsystolic shortening, and the scarred inferior and posterior segments show no circumferential contraction. GCS for the ventricle is depicted as a function of time on graph 114 by dashed white line. The solid lines on graph 114 are the local circumferential strains around the short axis cross section. Local Circumferential Strains for the left ventricle at the level in which the short axis plane was acquired are depicted as a spatial color map 116 with the colors being defined in color key 118.

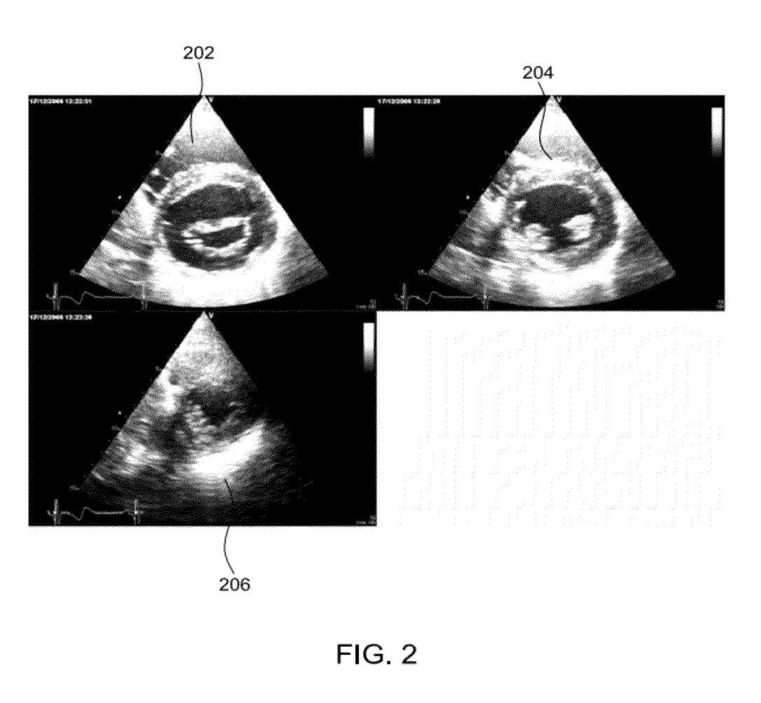

As described in the background, known techniques can use 2D ultrasound scans taken along the short axis (horizontal plane) of a left ventricle to determine GCS. FIG. 2 depicts three 2D ultrasound scans taken along the short axis (horizontal plane) of a left ventricle. Scan 202 is taken at the apex (AP) plane of the left ventricle. Scan 204 is taken at papillary muscle (PM) plane of the left ventricle. Scan 206 is taken at the mitral valve (MV) plane of the left ventricle.

In embodiments of the present technology, scans taken along the long axis (vertical plane) of a structure, such as the left ventricle, for example, can be used to determine GCS at any horizontal plane of the structure.

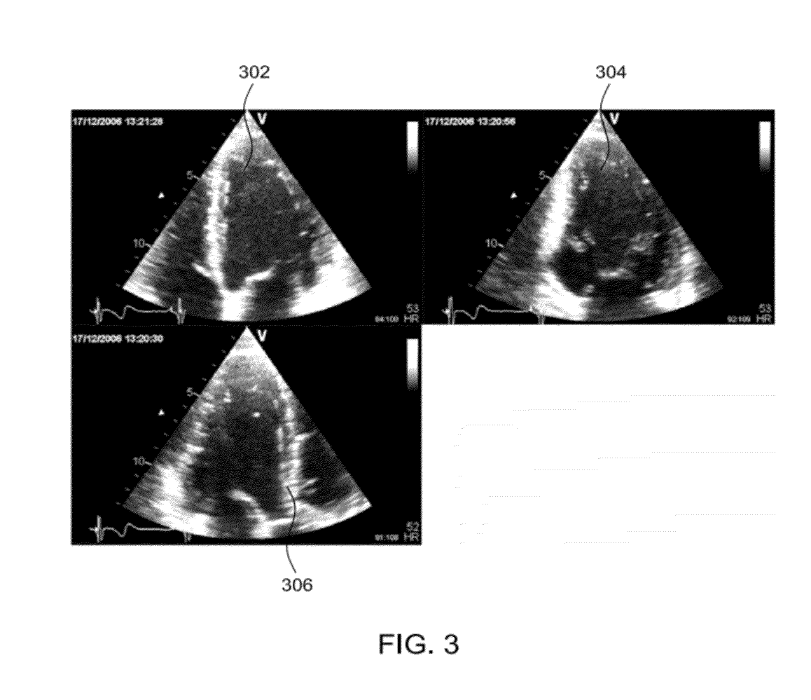

FIG. 3 depicts three 2D ultrasound scans taken along the long axis (vertical plane) of a left ventricle. Scan 302 is taken about a first vertical plane. Scan 304 is taken about a second vertical plane. Scan 306 is taken about a third vertical plane.

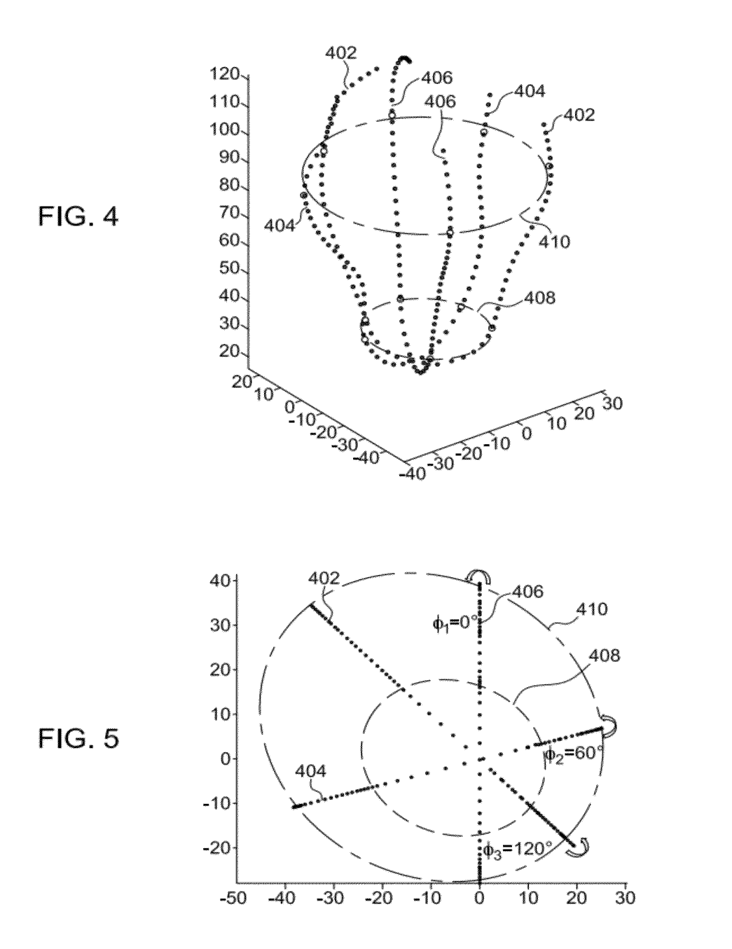

The outline of the left ventricle from each such scan can be identified and plotted as data points in a three dimensional graph, as depicted, for example, in FIG. 4. As shown in FIG. 4, data points 402 correspond to the outline of a left ventricle taken along a first vertical plane of the left ventricle. Data points 404 correspond to the outline of the same left ventricle taken along a second vertical plane. Data points 406 correspond to the outline of the same left ventricle taken along a third vertical plane. The three scans are taken at the same time so that the derived data points can accurately depict the volume of the structure at that time.

In the depicted embodiment, each vertical plane 402, 404, 406 is separated by sixty degrees. That is, as shown in FIG. 5, which depicts a two-dimensional top view of the data points depicted in FIG. 4, data points 406 lie on the vertical plane that corresponds with φ₁=0 degrees, data points 404 lie on the vertical plane that corresponds with φ₂=60 degrees, and data points 402 lie on the vertical plane that corresponds with φ₃=120 degrees.

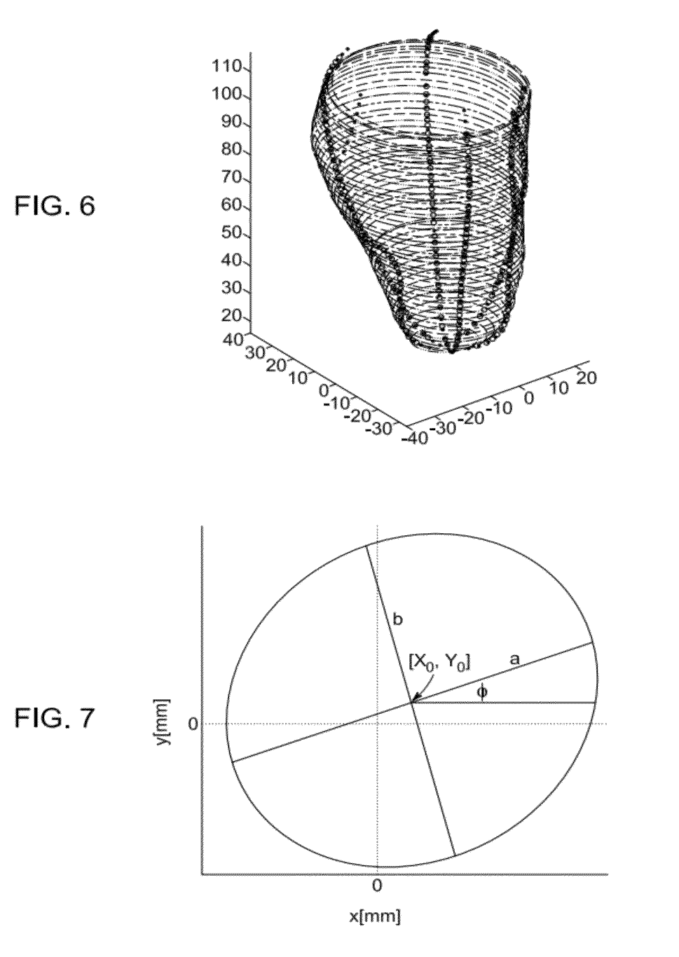

As shown in FIG. 4, for example, data points 402, 404 and 406 provide six data points at various horizontal planes of the depicted ventricle. Providing an ellipse through the six data points for any given horizontal plane can provide an estimated circumference for the ventricle at that horizontal plane. For example, FIGS. 4-5 depict ellipses 408 and 410 at different horizontal planes of the depicted ventricle, and FIG. 6 depicts various ellipses at different horizontal planes of the depicted ventricle.

The circumference (C) (in units of length) of each ellipse can be determined using a known estimation technique, such as the following equation, for example, where the values of a and b are the lengths of line segments a and b depicted in FIG. 7.

C≈π[3(a+b)−\sqrt{(3a+b)(a+3b)}]

GCS (ε) (in %) can then be determined using the following equation, where Cir₀ is the circumference at a given horizontal plane at the beginning of systole (contraction) and Cir is the circumference at the same horizontal plane at another point in time. The maximal GCS is generally identified as being at the end of systole (contraction).

$$ɛ = \frac{Cir - {Cir}_{0}}{{Cir}_{0}}$$

An example GCS calculation for an embodiment is as follows. For a horizontal slice located at z=65 mm, which is located at the papillary muscles (PM) level, the ellipse estimation at the image frame corresponding to the beginning of systole provides: a=36.56, b=33.26, phi=−0.26, X0=8.30, Y0=−1.44, which corresponds to a Cir₀ of 219.56 mm. For the same horizontal slice, the ellipse estimation at the image frame corresponding to the end of systole provides: a=28.69, b=30.30, phi=0.49, X0=9.14, Y0=4.5269, which corresponds to a Cir of 185.35 mm. Applying the GCS equation yields a GCS of (185.35−219.56)/219.56×100−15.58%.

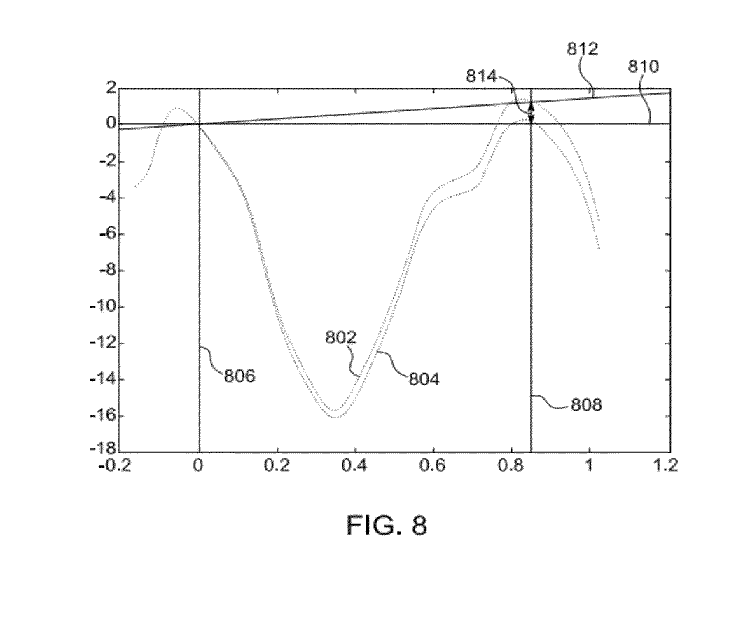

In certain embodiments, it can be assumed that the circumference of the ventricle at the beginning and end of a heart's cycle is identical thus, the strain at the end of the heart cycle shall be zero, which can allow drift compensation to be applied in order to improve accuracy of estimated circumference, and thus improve accuracy of the GCS calculation. FIG. 8 depicts a technique for correcting circumference strain estimates in accordance with embodiments of the present technology. Line 802 indicates circumference strain estimates calculated as described above. Beginning of the heart's cycle is indicated by line 806 and end of the heart's cycle is indicated by line 808. Line 812 is provided between the circumference strain estimates at the beginning and end of the heart's cycle. Line 810 is provided through the circumference strain value at the beginning of the heart's cycle and has a slope of zero, such that it is provided through zero circumference strain at the end of the heart's cycle. The vertical distance 814 between lines 810 and 812 represents the drift between the circumference strain estimate at the beginning of the heart's cycle and at the end of the heart's cycle. In the example, the estimate has drifted up to a higher circumference than was estimated at the beginning of the heart's cycle. Thus, if it is assumed that the circumference at the beginning and end of the heart's cycle are the same, the circumference strain estimate can be decreased by an amount equal to vertical distance 814 in order to correct for drift. This correction can be applied linearly to correct for drift at any time between the beginning and end of the heart's cycle, as less correction may be suitable at an earlier time in the cycle. The same correction can also be used to correct for downward drift.

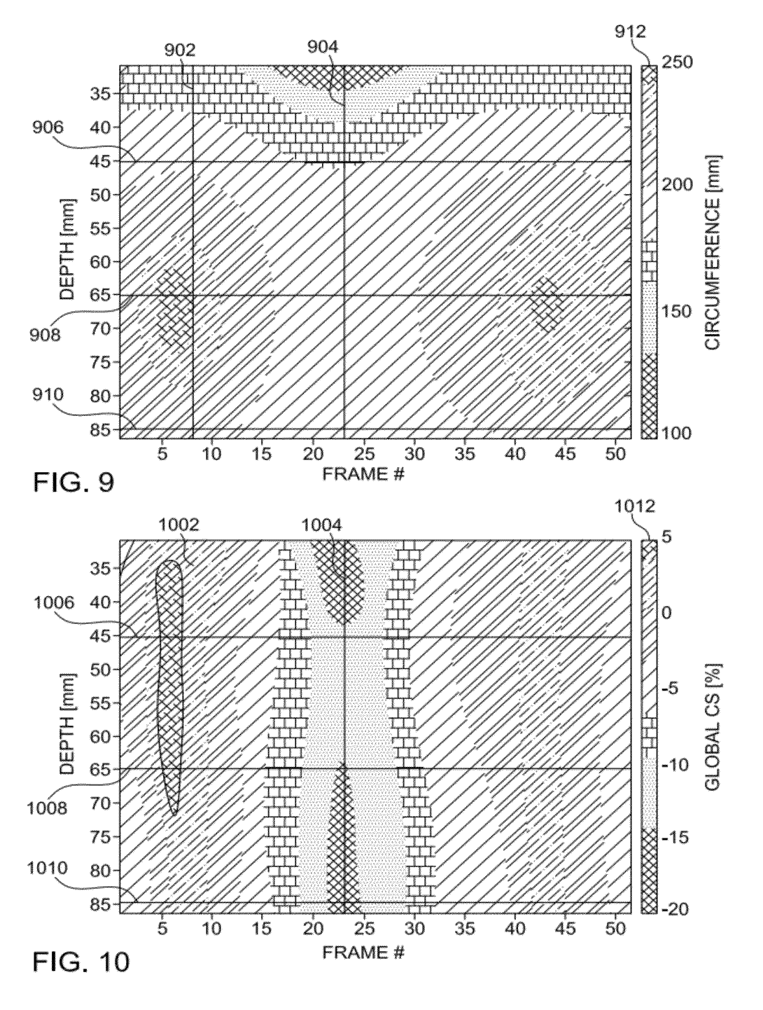

FIG. 9 depicts a color map that provides the circumference of a left ventricle over time in accordance with embodiments of the present technology. Line 902 indicates the beginning of systole. Line 904 indicates the end of systole. Line 906 corresponds to the horizontal AP plane. Line 908 corresponds to the horizontal PM plane. Line 910 corresponds to the horizontal MV plane. The color map corresponds to color key 912. In certain embodiments, circumference estimates determined as provided herein can be displayed to a user as a color map, as depicted, for example, in FIG. 9.

FIG. 10 depicts a color map that provides the GCS of a left ventricle over time in accordance with embodiments of the present technology. Line 1002 indicates the beginning of systole. Line 1004 indicates the end of systole. Line 1006 corresponds to the horizontal AP plane. Line 1008 corresponds to the horizontal PM plane. Line 1010 corresponds to the horizontal MV plane. The color map corresponds to color key 1012. In certain embodiments, GCS estimates determined as provided herein can be displayed to a user as a color map, as depicted, for example, in FIG. 10.

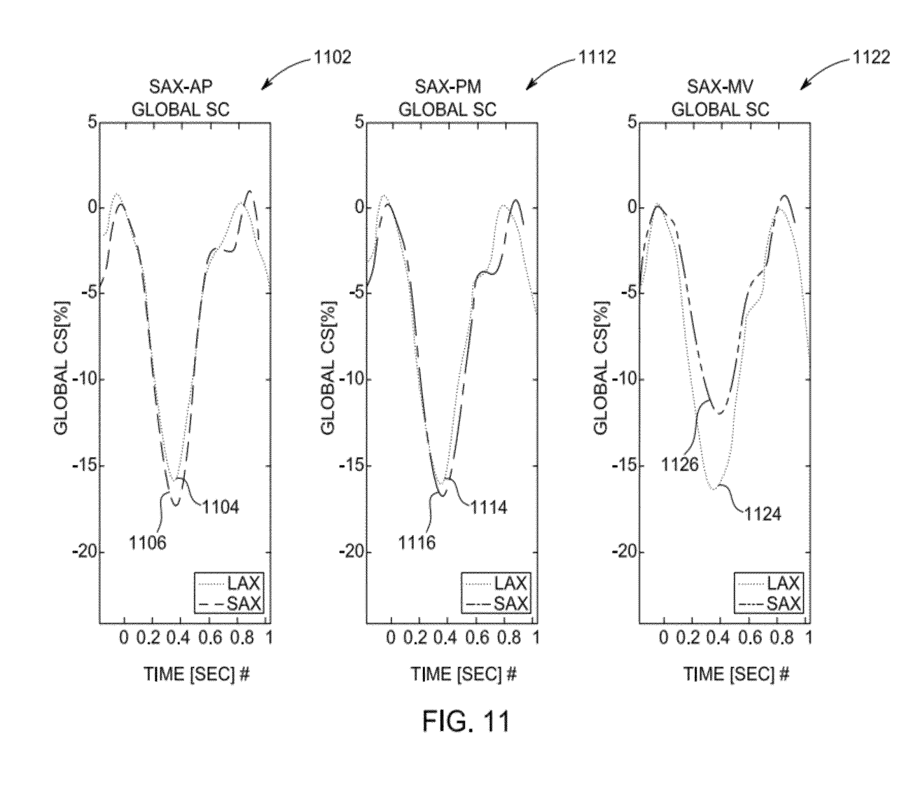

FIG. 11 depicts a comparison of GCS values determined using a known technique to GCS values determined using a technique in accordance with an embodiment of the present technology. Graph 1102 depicts GCS values at a horizontal AP plane of left ventricle as a function of time. Line 1104 indicates GCS estimates made using the long axis (vertical plane) techniques described herein. Line 1106 indicates GCS values provided using known short axis (horizontal plane) techniques.

Graph 1112 depicts GCS values at a horizontal PM plane of the left ventricle as a function of time. Line 1114 indicates GCS estimates made using the long axis (vertical plane) techniques described herein. Line 1116 indicates GCS values provided using known short axis (horizontal plane) techniques.

Graph 1122 depicts GCS values at a horizontal MV plane of the left ventricle as a function of time. Line 1124 indicates GCS estimates made using the long axis (vertical plane) techniques described herein. Line 1126 indicates GCS values provided using known short axis (horizontal plane) techniques.

As shown in FIG. 11, GCS estimates made using the long axis (vertical plane) techniques described herein are comparable, and sometimes identical, to GCS values provided using known short axis (horizontal plane) techniques. Thus, the present techniques can be used in place of known techniques to provide suitable GCS estimates without certain drawbacks associated with known techniques.

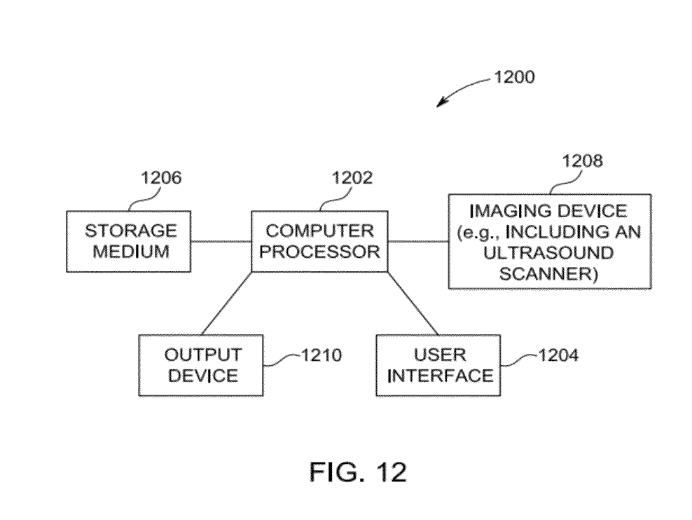

Embodiments of the present technology can be implemented in connection with a clinical information system and/or an ultrasound imaging system, for example. As depicted in FIG. 12, certain embodiments are implemented using a system 1200 that includes a computer processor 1202 in operable communication with a user interface 1204, a storage medium 1206, an imaging device 1208, and an output device 1210. In certain embodiments, the components of system 1200 can be implemented in any combination, such as a single integrated device, or as stand-alone components in operable communication, for example.

Processor 1202 can be configured to execute instructions encoded on storage medium 1206 and/or on another computer-readable medium. Processor 1202 can be configured to facilitate communication among user interface 1204, storage medium 1206, imaging device 1208, and output device 1210. Processor 1202 can execute instructions using information from user interface 1204, storage medium 1206, imaging device 1208, output device 1210 and/or other software applications to: (1) calculate the circumference of a structure and/or GCS using the techniques described herein, and/or (2) provide circumference and/or GCS analysis outputs, such as line graphs and/or color maps, as described herein.

User interface 1204 can be configured to allow commands to be input by a user. User interface 1204 can include a keyboard, mouse, switches, knobs, buttons, track ball, touch-screen, microphone configured to receive voice-activated commands and/or on screen menus, for example. In certain embodiments, user interface 1204 can be configured to allow a user to select a long axis (vertical) plane to be used to estimate circumference and/or GCS using the techniques described herein. In certain embodiments, user interface 1204 can be configured to allow a user to select a horizontal plane at which to estimate circumference and/or GCS using the techniques described herein. In certain embodiments, user interface 1204 can be configured to allow a user to select an output format for circumference and/or GCS analysis, such as line graphs and/or color maps, for example.

Storage medium 1206 can be any tangible, non-transitory computer-readable medium that is readable by processor 1202, whether local, remote, connected by wires and/or connected wirelessly. For example, storage medium 1206 can include a computer hard drive, a server, a CD, a DVD, a USB thumb drive, and/or any other type of tangible memory capable of storing one or more computer instructions. The sets of instructions can include one or more routines capable of being run or performed by processor 1202.

Imaging device 1208 can be configured to capture an image of a structure, such as a left ventricle, for example. Imaging device 1208 can use ultrasound, x-ray, computed tomography and/or any other imaging modality to capture an image of the structure. In certain embodiments, imaging device 1208 can be configured to automatically select a long axis (vertical) plane to be used to estimate circumference and/or GCS using the techniques described herein. In certain embodiments, imaging device 1208 can be configured to automatically select a horizontal plane at which to estimate circumference and/or GCS using the techniques described herein. In certain embodiments, imaging device 1208 can be an ultrasound scanner.

Output device 1210 can be configured to output information from system 1200, and can comprise any device suitable for this task. In certain embodiments, for example, output device 1210 can output a visual display of a structure captured by imaging device 1208, and circumference and/or GCS information, such as depicted in FIGS. 1 and 9-10, for example. In certain embodiments, for example, output device 1210 can comprise a computer monitor, liquid crystal display screen, printer, fax machine, e-mail server and/or speaker, for example.

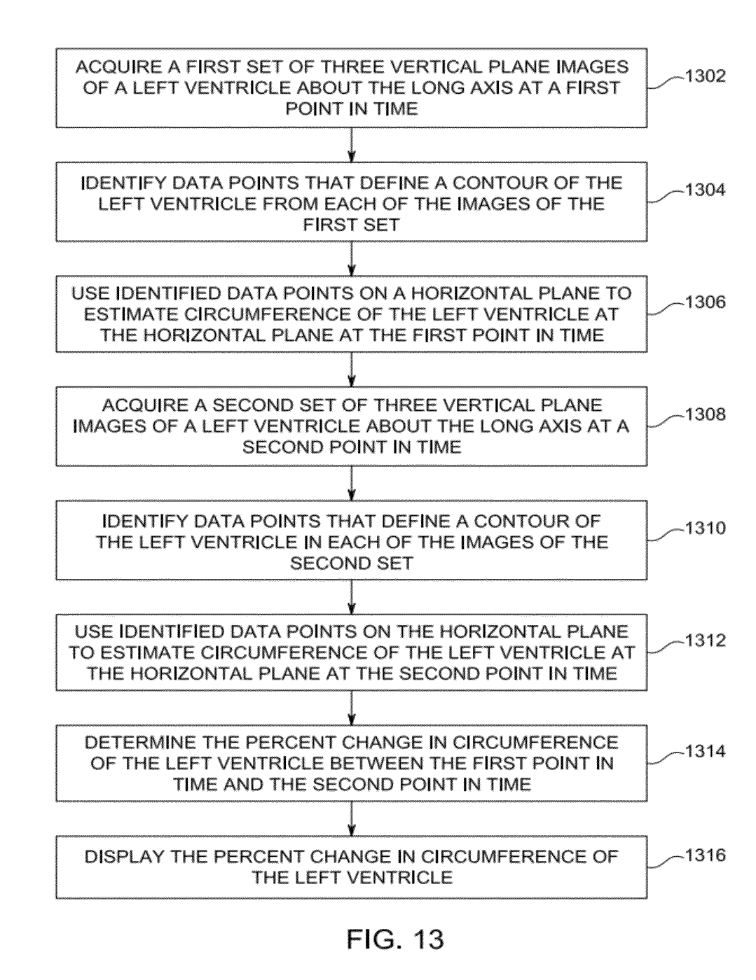

FIG. 13 depicts a method 1300 for determining GCS in accordance with embodiments of the present technology. The method can be applied by employing the techniques and systems described herein. At 1302, a first set of three vertical plane images of a left ventricle about the long axis at a first point in time is acquired. At 1304, data points that define a contour of the left ventricle from each of the images of the first set are identified. At 1306, identified data points on a horizontal plane are used to estimate circumference of the left ventricle at the horizontal plane at the first point in time. At 1308, a second set of three vertical plane images of a left ventricle about the long axis at a second point in time is acquired. At 1310, data points that define a contour of the left ventricle in each of the images of the second set are identified. At 1312, identified data points on the horizontal plane are used to estimate circumference of the left ventricle at the horizontal plane at the second point in time. At 1314, the percent change in circumference of the left ventricle between the first point in time and the second point in time (GCS) is determined. At 1316, the percent change in circumference of the left ventricle (GCS) is displayed.

In certain embodiments, a method can further include applying drift compensation as described in connection with FIG. 8, for example. In such embodiments, drift compensation may improve accuracy of estimated circumference and/or GCS.

Certain embodiments of the present invention may omit one or more of these steps and/or perform the steps in a different order than the order listed. For example, some steps may not be performed in certain embodiments of the present invention. As a further example, certain steps may be performed in a different temporal order, including simultaneously, than listed above.

One or more of the steps of the method 1300 may be implemented alone or in combination in hardware, firmware, and/or as a set of instructions in software, for example. Certain embodiments may be provided as a set of instructions residing on a tangible, non-transitory computer-readable medium, such as a memory, hard disk, DVD, or CD, for execution on a general purpose computer or other processing device. For example, certain embodiments provide a computer-readable storage medium encoded with a set of instructions for execution on a processing device and associated processing logic, wherein the set of instructions includes a routine(s) configured to provide the functions described in connection with the method 1300.

Applying the method 1300 as described above, and/or in light of the techniques and systems described herein, can provide a technical effect of estimating circumference and/or GCS of a left ventricle at any horizontal plane of the left ventricle without requiring short axis (horizontal plane) scans of the left ventricle.

Certain image data acquired, analyzed and displayed in connection with the techniques described herein represent human anatomy, such as a left ventricle, for example. In other words, outputting a visual display based on such data comprises a transformation of underlying subject matter (such as an article or materials) to a different state.

While the inventions have been described with reference to embodiments, it will be understood by those skilled in the art that various changes may be made and equivalents may be substituted without departing from the scope of the inventions. In addition, many modifications may be made to adapt a particular situation or material to the teachings of the inventions without departing from their scope. Therefore, it is intended that the inventions not be limited to the particular embodiments disclosed, but that the inventions will include all embodiments falling within the scope of the appended claims.

1. A method for determining circumference and a percent change in circumference of a structure comprising:
receiving at a processor a first set of at least three vertical plane images of a structure about a long axis of the structure that were acquired at a first point in time;
using the processor to identify data points from each of the images of the first set that define a contour of the structure;
using the processor to estimate circumference of the structure at a horizontal plane of the structure at the first point in time using identified data points positioned on the horizontal plane;
receiving at a processor a second set of at least three vertical plane images of a structure about a long axis of the structure that were acquired at a second point in time;
using the processor to identify data points from each of the images of the second set that define a contour of the structure;
using the processor to estimate circumference of the structure at the horizontal plane of the structure at the second point in time using identified data points positioned on the horizontal plane; and
using the processor determine the percent change between the estimated circumference of the structure at the first point in time and the estimated circumference of the structure at the second point in time.

2. The method of claim 1, wherein the structure is a left ventricle and the percent change between the circumference of the structure at the first point in time and at the second point in time is a global circumferential strain for the left ventricle.

3. The method of claim 2, further comprising displaying the global circumferential strain as a color map.

4. The method of claim 2, further comprising displaying the global circumferential strain as a line chart.

5. The method of claim 2, further comprising adjusting at least one of the estimated circumferences of the structure using a linear drift correction that assumes the circumference of the left ventricle is the same at the beginning and end of each heart pump cycle.

6. The method of claim 2, wherein the first point in time is the beginning of systole, and wherein the second point in time is the end of systole.

7. The method of claim 1, wherein the vertical plane images are acquired using an ultrasound scanner.

8. A system for determining circumference and a percent change in circumference of a structure comprising:
a processor configured to receive a first set of at least three vertical plane images of a structure about a long axis of the structure that were acquired at a first point in time,
the processor configured to identify data points from each of the images of the first set that define a contour of the structure,
the processor configured to estimate circumference of the structure at a horizontal plane of the structure at the first point in time using identified data points positioned on the horizontal plane,
the processor configured to receive a second set of at least three vertical plane images of a structure about a long axis of the structure that were acquired at a second point in time,
the processor configured to identify data points from each of the images of the second set that define a contour of the structure,
the processor configured to estimate circumference of the structure at the horizontal plane of the structure at the second point in time using identified data points positioned on the horizontal plane, and
the processor configured to determine the percent change between the estimated circumference of the structure at the first point in time and the estimated circumference of the structure at the second point in time.

9. The system of claim 8, wherein the structure is a left ventricle and the percent change between the circumference of the structure at the first point in time and at the second point in time is a global circumferential strain for the left ventricle.

10. The system of claim 9, further including an output device configured to display the global circumferential strain as a color map.

11. The system of claim 9, further including an output device configured to display the global circumferential strain as a line chart.

12. The system of claim 9, wherein the processor is configured to adjust at least one of the estimated circumferences of the structure using a linear drift correction that assumes the circumference of the left ventricle is the same at the beginning and end of each heart pump cycle.

13. The system of claim 9, wherein the first point in time is the beginning of systole, and wherein the second point in time is the end of systole.

14. The system of claim 8, further including an ultrasound scanner configured to acquire the vertical plane images.

15. A non-transitory computer-readable medium encoded with a set of instructions for a computer, the instructions comprising:
a first routine configured to receive a first set of at least three vertical plane images of a structure about a long axis of the structure that were acquired at a first point in time;
a second routine configured to identify data points from each of the images of the first set that define a contour of the structure;
a third routine configured to estimate circumference of the structure at a horizontal plane of the structure at the first point in time using identified data points positioned on the horizontal plane;
a fourth routine configured to receive a second set of at least three vertical plane images of a structure about a long axis of the structure that were acquired at a second point in time;
a fifth routine configured to identify data points from each of the images of the second set that define a contour of the structure;
a sixth routine configured to estimate circumference of the structure at the horizontal plane of the structure at the second point in time using identified data points positioned on the horizontal plane; and
a seventh routine configured to determine the percent change between the estimated circumference of the structure at the first point in time and the estimated circumference of the structure at the second point in time.

16. The medium and instructions of claim 15, wherein the structure is a left ventricle and the percent change between the circumference of the structure at the first point in time and at the second point in time is a global circumferential strain for the left ventricle.

17. The medium and instructions of claim 16, further including an eighth routine configured to display the global circumferential strain as a color map.

18. The medium and instructions of claim 16, further including an eighth routine configured to display the global circumferential strain as a line chart.

19. The medium and instructions of claim 16, further including an eighth routine configured to adjust at least one of the estimated circumferences of the structure using a linear drift correction that assumes the circumference of the left ventricle is the same at the beginning and end of each heart pump cycle.

20. The medium and instructions of claim 16, wherein the first point in time is the beginning of systole, and wherein the second point in time is the end of systole.
